(19) United States
(12) Patent Application Publication
(10) Pub. No.: US 2010/0198175 A1
(43) Pub. Date: Aug. 5, 2010

(54) PORTABLE DRAINAGE SYSTEM WITH INCORPORATED SUCTION

(76) Inventor: German Rosero, Pasto (CO)

Correspondence Address: SANCHELIMA & ASSOCIATES, P.A.;JESUS SANCHELIMA, ESQ., 235 S.W. LE JEUNE ROAD, MIAMI, FL, 33134, US

(21) Appl. No.: 12/365,972

(22) Filed: Feb. 5, 2009

(51) Int. Cl. A61M 1/00

(52) U.S. Cl. 604/320

(57) ABSTRACT

A drainage system comprising a portable ergonomic carrier, the carrier further comprising at least one electric motor which moves a moving member that is powered by at least one battery and that is commanded by at least one electronic driver. The drainage system also includes a disposable collector that comprises at least one suction mechanism or pump that is adapted to receive said moving member and this collector also includes a least one valve mechanism and at least one filter mechanism. The collector forms at least one collecting chamber and is engagingly mated and coupled demountably into the portable ergonomic carrier.

### FIELD OF THE TECHNOLOGY

The present invention relates to suction and drainage devices used to drain liquids and gases from the human body.

### BACKGROUND OF THE TECHNOLOGY

Normally, the pressure in the lungs is greater than the pressure in the pleural space surrounding the lungs. However, if air enters the pleural space, the pressure in the pleura then becomes greater than the pressure in the lungs, causing the lung to collapse partially or completely. Pneumothorax can be either spontaneous or due to trauma. If a pneumothorax occurs suddenly or for no known reason, it is called a spontaneous pneumothorax. This condition most often strikes tall, thin men between the ages of 20 to 40. In addition, people with lung disorders, such as emphysema, cystic fibrosis, and tuberculosis are at higher risk for spontaneous pneumothorax. Traumatic pneumothorax is the result of accident or injury due to medical procedures performed to the chest cavity, such as thoracentesis or mechanical ventilation. Tension pneumothorax is a serious and potentially life-threatening condition that may be caused by traumatic injury, chronic lung disease, or as a complication of a medical procedure. In this type of pneumothorax, air enters the chest cavity, but cannot escape. This greatly increased pressure in the pleural space causes the lung to collapse completely, compresses the heart, and pushes the heart and associated blood vessels toward the unaffected side.

Pumps for pleural drainage have inter alia contributed to the reduction of mortality and a solution to the aforementioned problems. Compact drainage systems for pleural drainage have been used and these devices have included collection chambers with a water seal which prevents the returning of the air to the pleural cavity and connected to a suction system. Nevertheless, there are several issues and problems with said compact systems. In the medical community for example, it would be desirable to progress from systems where in order to keep the water seal, it is necessary a column of water of some height and a float valve in the water seal chamber to systems for indicating the respiration of the patient and a bubble chamber which indicates the passage of air bubbles from the pleural cavity to the suction source. These systems are usually large and cumbersome for patients that would like to move around.

Furthermore, the medical community has in the past relied on systems with a disposable component containing the collection chambers, attached to a non-disposable fixed systems as well as fixed suction regulators and valves. Here again, the issue with this type of designs is that the disposable chambers are not adapted to the human body. Patient's mayor complaint relies in the non-ergonomic design that makes it uncomfortable and unbearable. The non-disposable system has always been attached to the hospital walls forcing patients to stay connected and in bed to avoid complications.

There is also need in the industry, to manufacture pleural drainage devices include multiple advanced tools for diagnoses useful for monitoring of patients as well a portable system so that the patient is free to move. For example in the thoracostomy medical arts, there is a need to provide the patient who requires a closed thoracostomy, a portable drainage system, which is ergonomic and comfortable. This ergonomic mobile system would create faster recovery of the patient as well as the important reduction of the hospital expenses avoiding prolonged hospitalizations.

Moreover, in the drainage system arts, it would be desirable to progress from the fixed to the wall system that use an anchored suction mechanism, to an ergonomic suction mobile device with an insitu pumping and valve source. Fixed systems, in many cases are unnecessary in clinically stable patients, which promote complications because of confinement to resting in bed due to the traditional management of his pleural pathology. They are forced to be connected to uncomfortable drainage devices, anchored to a wall suction dependant of a central pump source at the hospital.

There is also a further need in the medical device arts for a portable disposable drainage system that generates permanent suction in an autonomous manner without the necessity of connecting to a suction external source (which generally is connected to the wall) and so, giving the patient the convenience, freedom and safety of movement, early ambulation and a possible ambulatory management of his pathology when indicated.

### BRIEF SUMMARY OF THE TECHNOLOGY

The present technology provides an ergonomic, portable drainage system which includes an ergonomic carrier and a disposable collection apparatus with incorporated suction pump.

### BRIEF DESCRIPTION OF THE SEVERAL VIEWS OF THE DRAWINGS

The foregoing summary, as well as the following detailed description of the technology, will be better understood when read in conjunction with the appended drawings. For the purpose of illustrating the technology, there are shown in the embodiments which are presently preferred. It should be understood, however, that the technology is not limited to the precise arrangements and instrumentalities shown. In the drawings:

FIG. 1 depicts the portable ergonomic carrier apparatus.

FIG. 2 depicts the disposable collector apparatus.

FIG. 3 depicts an alternative means to carry the portable ergonomic carrier using straps.

FIG. 4 depicts the portable ergonomic carrier further depicting the cover that hides the inner workings of the motor, battery, LED, electronic board and electric charger.

FIG. 5 depicts the portable ergonomic carrier with interior battery, electronic controller, the motor and the connecting rod or external moving member.

FIG. 6 depicts an embodiment of the technology, where diaphragm pump is used and dumbbell valves, as suction mechanism.

FIG. 7 depicts an embodiment of the technology, where a piston pump is used and ball valves, as suction mechanism.

FIG. 8 depicts an embodiment of the technology, where a fluid mover type pump is used and a single ball valve, as suction mechanism.

FIG. 9 depicts an embodiment of the technology, where the internal pump system is composed of gears to move the fluids.

FIG. 10 depicts an embodiment of the technology, where the internal pump system is composed of a propeller to move the fluids.

FIG. 11 depicts the respiration monitor and the water trap as filtering mechanism.

FIG. 12 depicts an embodiment of the technology, where a piston pump is used as suction mechanism, further depicting the engagement of the detachable moving member coming from the motor. It further depicts two valves to control the forward flow of fluids thought the system.

FIG. 13 depicts an embodiment of the technology, in 3D in which the portable ergonomic carrier apparatus hides the disposable collector apparatus and the quantity of fluid can be seen though windows. It further depicts a thorax-tube that the system is attached to and the battery charging means.

FIG. 14 depicts how the ergonomic design of the device helps its adaptation to the patient, making it comfortable when installing and permitting a wide range of movements.

### DESCRIPTION OF THE TECHNOLOGY

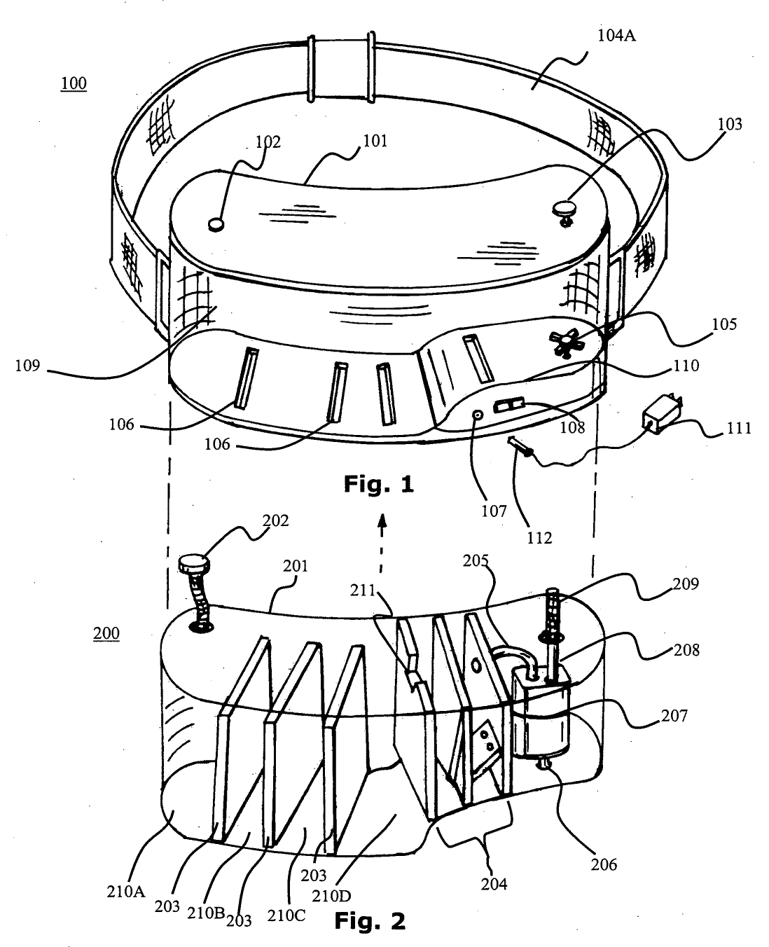

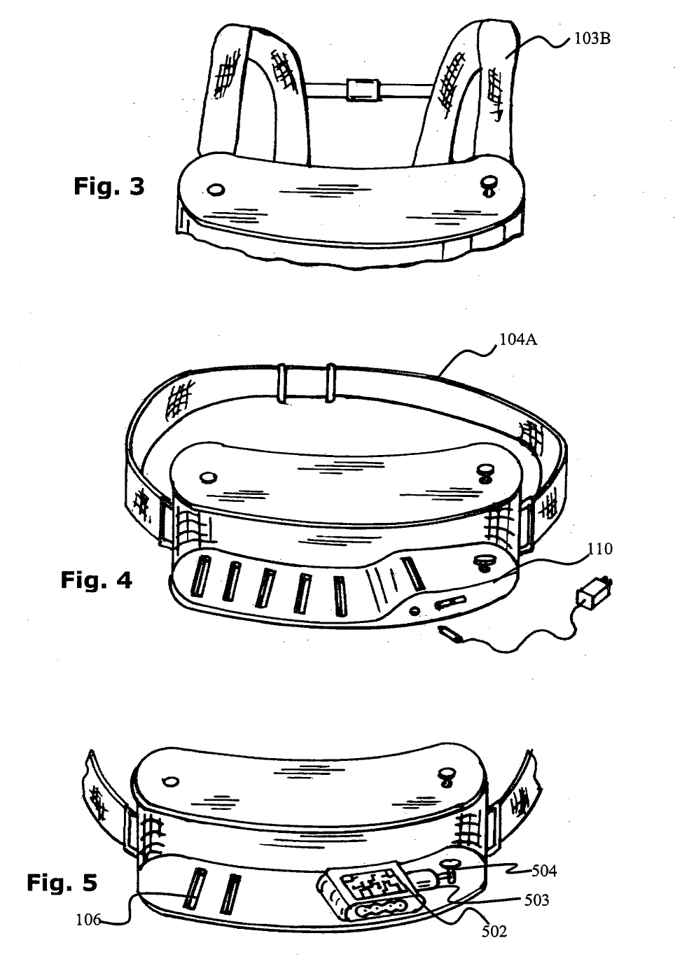

The present technology provides for an ergonomic, portable drainage system. Said system provides for a portable ergonomic carrier apparatus depicted in FIG. 1, and a disposable collector apparatus FIG. 2. Said disposable collector 200 is engagingly mated and coupled demountably into said portable ergonomic carrier 100. The disposable collector 200 is to be replaced and discarded when it is filled with liquids drained from a human body. The present technology is the solution to the aforementioned problem in that the system is hygienic in that the once the collector is used can be thrown away. Also, that the carrier 100 apparatus is portable and can be transported. Without limiting the scope of the invention to pleural drainage or human thoracotomies, the technology can also be used to drain fluids from a dead person for embalmment or even an animal.

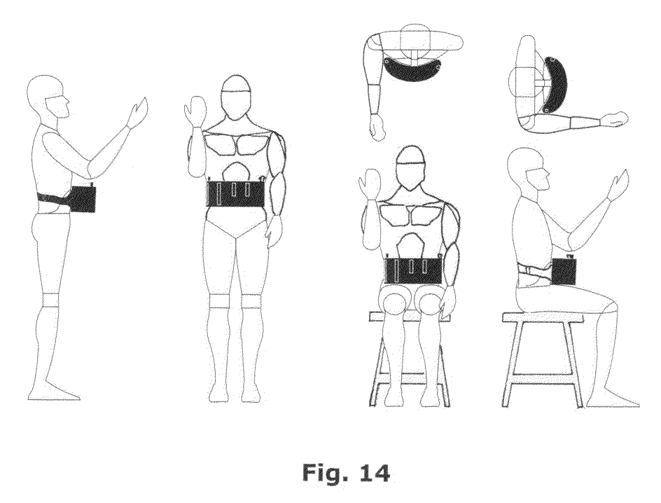

FIG. 1, depicts at least one preferred method of manufacture said ergonomic carrier apparatus 100. The carrier 100 is made to be portable which is intended to be carried or moved with ease, easily or conveniently transported and not fixed as shown in FIG. 14. It further comprises an ergonomic design which is adapted to the human body or intended to maximize recovery by minimizing patient fatigue and discomfort. Said carrier apparatus 100 further comprises a frame structure 101 that defines a cavity, said frame structure material is selected from the group consisting of, stamped or bent metal such as aluminum, formed plastic polymers which further include acrylics, polyesters, silicones, polyurethanes, and halogenated plastics. The frame 101 of the carrier is attached to the human body by means of straps 104B or at least one belt 104A. The ergonomic design of the device helps its adaptation to the patient, making it comfortable when installing and permitting a wide range of movements as shown in FIG. 14.

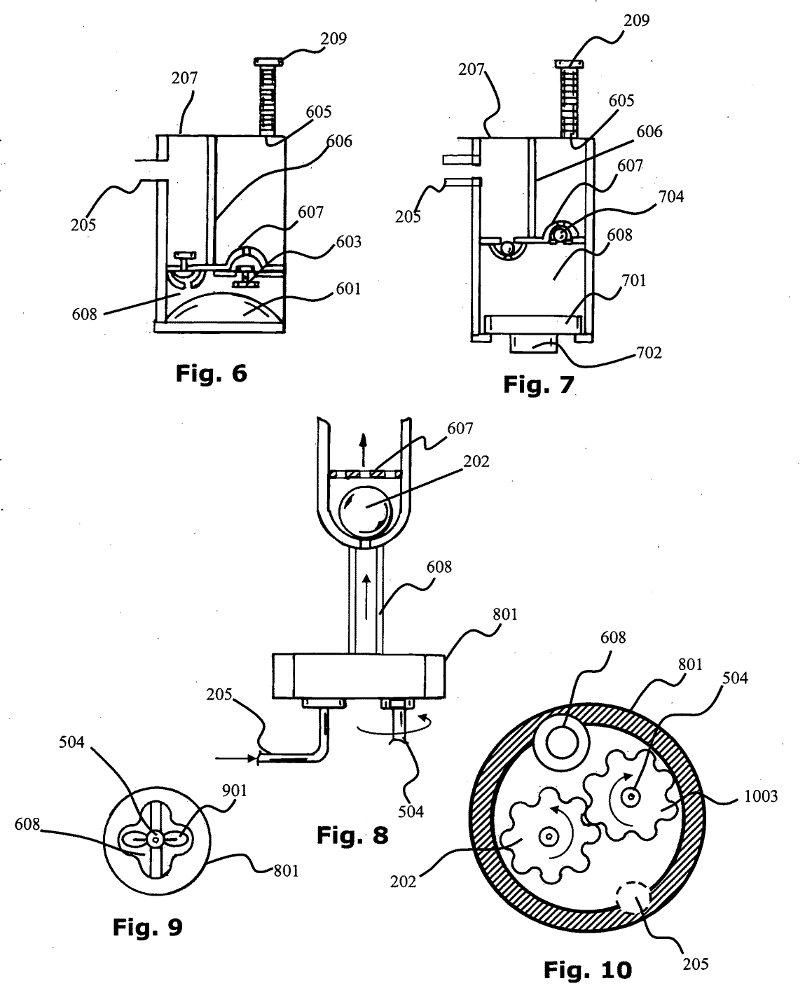

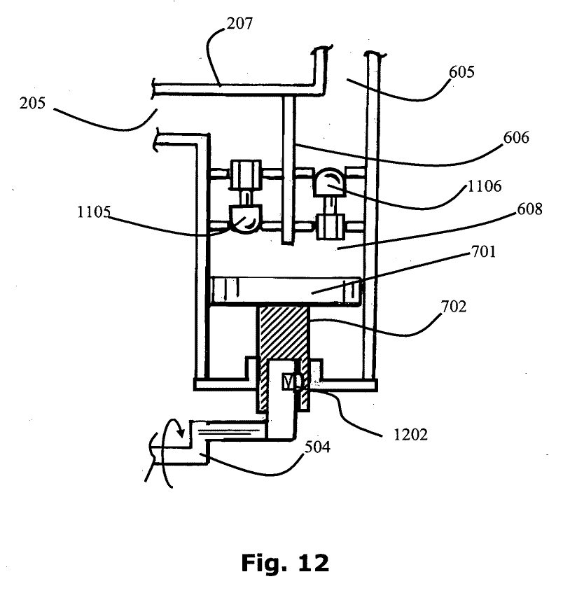

The ergonomic carrier apparatus 101 further comprises at least one electric motor 503 or similar device, designed to create pressure in order to pump the gasses and liquids from the patient. Said electric motor is equivalent to means to accurately control the movement of an object based on speed, distance, load, inertia or a combination of all these factors. Said electric motor 503 can be selected from the group consisting of Stepper Motor, Linear Step Motor, DC Brush, Brushless, Servo, Brushless Servo and more. Said electric motor 503 advantageously creates circular or vertical motions to a moving member 504 attached. Said moving member 504 is further engagingly coupled demountably though 1202 to at least one suction mechanism 207 as depicted in FIG. 6, FIG. 7, FIG. 8 and FIG. 12. Said moving member 504 may be advantageously engagingly coupled demountably to a diaphragm mechanism 601 or a piston mechanism 701, attached by means of a receiving engagement member 702. Said moving member 504 may also engage a in a pump chamber 801 to move the pumping mechanisms depicted in FIG. 9 and FIG. 10. The moving member 504 may be used to move a two small gears such as 1003 and 1004 and the fluid would move in through 205 and out trough a chamber 608 before expulsion trough at least one valve. As an alternative embodiment, a fluid mover FIG. 9 using a propeller type pump with blades 901 can also be used for substantially same purpose to obtain the same pumping result.

The ergonomic carrier apparatus 100 further comprises at least a battery or battery pack 501 stringed in parallel or series to provide for the energy required to drive the electric motor 503. Said battery 501 is advantageously a rechargeable battery charged using external charger 111. The carrier apparatus 100 further comprises at least one electronic driver 502 further comprised of semiconductor and or a printed wiring board technology. Said driver 502 is meant to drive the battery, LED 108 and to make sure for the continuous operation of said motor 503. The electronic driver 502 is used to alternate the current to drive the electric motor and also to pulse the power to the motor 503 in order to conserve energy and to maintain a stable pumping rhythm. In at least one preferred embodiment, an indexer or controller microprocessor was used to generate step pulses and direction signals for the driver 502.

The disposable collector apparatus of FIG. 2 comprises at least one suction mechanism 207 that is adapted demountably to receive said moving member 504 with a receiving engagement member such as the one depicted 702. Several suction mechanisms can be used such as the ones depicted in FIG. 6, FIG. 7, FIG. 8 and FIG. 12. In at least one preferred embodiment consist of FIG. 12 a suction mechanism conformed by a chamber 608 and one-way valves 1105 and 1106, which permits the suction through at least one collecting chamber 210 A-D made by partitions 203 which permits the exit of air to the atmosphere guaranteeing continuous suction. Continuous suction is needed in order to keep constant the internal pressure and to avoid the re-entry of the air to the patient's pleural space. In at least one preferred embodiment, said one-way valves 1105 and 1106 are oriented in vertical form and one against the other in order to keep its function. These are composed by a an axis with a seal in one end to make the close hermetic in one way, and in the other side, four perpendicular “wings” to let the free passage of the air within its valve chamber in that same way. Said valves avoid the extracted air from the compartments 210 A-D return to the pleural cavity and minimize the risk of causing tension pneumothorax in the patient. An alternative embodiment is a different type of valve to be used such as “dumbbell” type 603 or a “ball” type valve such as 704, both may be used for substantially same purpose to obtain the same sealing result.

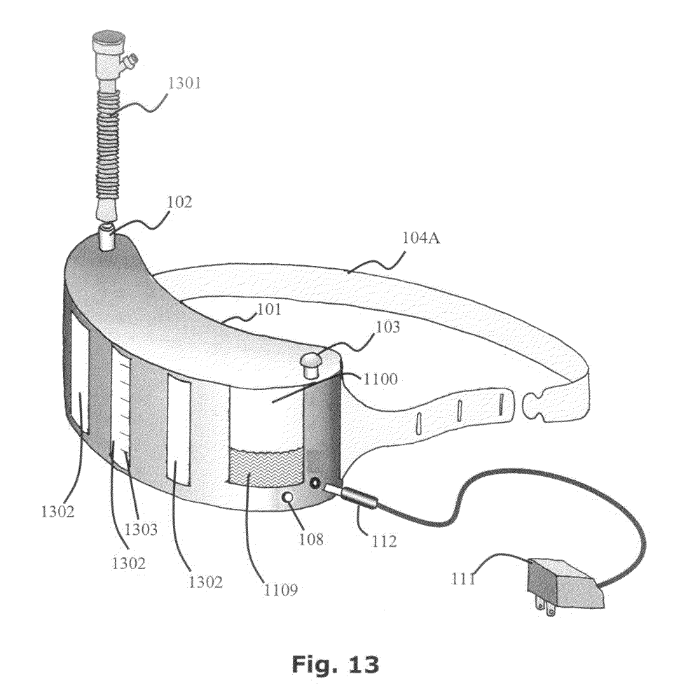

Said disposable collector apparatus 200 is advantageously made out of transparent or clear plastic in order to visualize and measure the drained liquid. The transparent plastic is selected from the group of polycarbonate, polymethylmethacrylate, polystyrene, styrene-acrylonitrile copolymer, and poly (vinyl chloride). Said clear plastic was painted with a scale 1303 in cc to visualize an exact measurement of the quantity of drained material when liquid. An alternative embodiment is depicted in FIG. 13 where the liquids are visualized from the exterior through small graduated windows 1302. Said plastic was molded into shape to create multiple collecting chambers 210A-D divided by barriers 203. FIG. 2 depicts a connector 202 adapted to a conventional thorax-tube 1301 and it is connected to a first collection chamber 210 in order to lodge de drained material from the patient's pleural space (blood, leaks, pus, air). Such chamber 210A is joined in its top part through an internal opening with the next collection chamber 210B and again with the others 210C and 210D. Each of the chambers is joined between themselves and designed in a way they are filled consecutively one after the other up to get about a total volume of 1200 cc.

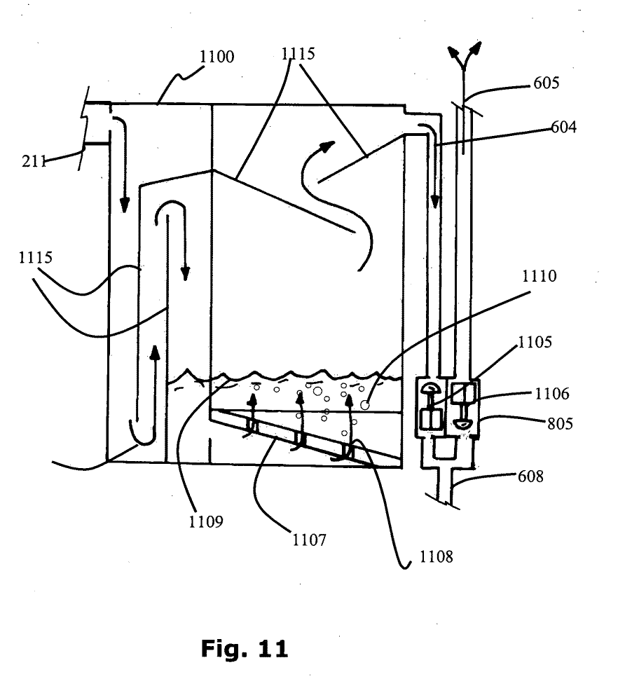

Said disposable collector apparatus 200 further comprises at least a filtering mechanism such as the one depicted in FIG. 11, this water trap or water seal 1100 as defined by the partitions 204. Compartments 204 define a respiration monitor is, which also provides for a water seal 1100 which traps the air extracted from the pleural cavity and from the inlet 202. Said water seal is one of the preferred filtering mechanisms which contains a bubble chamber 1107 that shows respiration or air/gas filtering though the transparent plastic the air passage 1108 coming through the chamber. Said water trap is usually filled with up to a defined height with water 1109 defining a bubbling chamber 1107. The chamber is provided with a pair of barriers 1115 to prevent the water from going into the suction valve 1105 when the patient makes a sudden movement. In alternative embodiments, other types of seals without water, using for example alcohols, gels, gelatins or oils may be used for substantially same purpose to obtain the same sealing result.

In at least one preferred embodiment, the negative pressure system was guaranteed by a stepping motor 503—of 9 volts, energized by a rechargeable battery 501 of same voltage. The motor 503 due to its internal system of gears reduces the revolutions until reach the 24 rpm and through the articulation with a connecting-rod or moving member 504 impulses an embolus 701, which is sealed in its distal side towards a suction chamber 608 forming so an autonomous system that provides negative pressure to a closed system for pleural drainage. The moving member 504 transmits the rotation movement with the embolus 701 engaged in 702. An alternative engagement is depicted in 100, where the mating part 105 engagingly mated and coupled demountably with its opposite side 206 in the pump by its distal side. The pump 207 is further comprised of a seal or valve pack 1105 and 1106. The suction chamber 608 sucks gases and liquids from the inlet 202 though the collecting chambers 210A-D though the water trap 1100 as defined by 204 and is further communicated with the valves 1105 and 1106, the gases finally exiting through exit tube 209 thus giving continuity to the system.

In alternative embodiment of the technology, the respiration monitor in the water trap 1100 is united with the suction system 207 through a tube 604 that comes from the exit of the one-way suction a single ball 704 valve such as the one defined in FIG. 8. A pumping mechanism enclosed in 801 with internal fluid movers such as in FIG. 10 and FIG. 9, move the fluids from the inlet tube 202, though 205 and finally thought the outlet tube 209. Said fluid mover mechanism of 801 is the one that transmits the negative pressure to the collection chambers 210 A-D and from them to the patient's pleural space. The fluid mover can be comprised of gears 1003 or propeller 901. The suction chamber 608 is opposite, a one-way leaking ball valve 704 with stopping mechanism 607 which permits the leaving of the accumulated air in the device into the atmosphere through the escape orifice 605 and tube 209, conserving so a constant internal pressure.

The pressure needed to be created by system thought the pump 207, needs to be enough to allow for normal breathing and at the same time enough pressure to extract gases and fluids from the pleural space. This is because pressure is greater than the lung to the pleura (this is a virtual cavity), the lung is covered with a membrane (the visceral pleura) and this membrane covers the inside of the thoracic cavity (parietal pleura). This virtual cavity has only a small amount of liquid, but the mechanics of breathing is like in a closed cavity. In constant temperature, the pressure of a closed cavity is inversely proportional to its volume P₂V₂. So when the thoracic cavity allows us to breathe, the diaphragm muscle descends and the rib cage is producing large fall in pleural pressure to about −10 (cm H₂O) allowing expansion of the lung, and the contrary, when the diaphragm rises, the pleural pressure is more negative. The distensibility of a normal lung is 200 (ml/cm H₂O) this is when there is normal breathing of 500 to 700 cm needed to lower the pressure in the pleura about −2.5 to −3.5 (cm H₂O). Without limiting the scope of the disclosed technology, this means that the intraplenural pressures created by the pump 207 need to be about −10 (cm H₂O) negative in order to allow for normal breathing to occur simultaneously.

In another embodiment of the technology FIG. 13, a universal connector 202 is joined to a thorax tube 1301 used in standard drainage procedures. Said connector 202 is further attached with four collection chambers 210A-D with about a capacity of 1000 cc, intercommunicated between them and then connected to a respiration monitor 1100 which integrates the water seal of about 2-cm height comprising a bubble chamber 1107 to indicate the passage of air from the pleural cavity. Preceding the monitor 1100 is attached with the one-way valve system 603 to a diaphragm 601 suction the air and then be liberated to the atmosphere, though 209. The motor 503, fed by a 6 volts rechargeable battery. Due to its internal system of gears which as well as reduces its revolutions, generates the necessary strength to realize its plunging movement with a minimal energy consumption, further articulated with its connecting-rod 504 which generates a circular movement into linear, transmitting it to the diaphragm 601 which moves the gases in the interior of the suction chamber 608.

FIG. 1 and FIG. 2 depict the drainage system in its entirety with its external components. The portable ergonomic carrier apparatus depicted in FIG. 1, and a disposable collector apparatus FIG. 2 or disposable collector 200 is engagingly mated and coupled demountably into said portable ergonomic carrier 100 using several female-male guided attachments 106. The guides 106 will engage and disengage when the carrier 101 is coupled with the disposable collector 201 for easy removal or detachment. The Cover 110 hides the battery 501, the electronic controller 501, the motor 503 and part of the connecting rod 504. There is at least one LED light 108 which lights when the device is working and the battery is charged, and besides it, a jack 107 that couples with male 112 end from charger 111 to charge battery and/or put the system to work through an external source of energy, guaranteeing the continuous functioning of the system although the battery is charging.

It will be appreciated by those skilled in the art that changes could be made to the embodiments described above without departing from the broad inventive concept thereof. It is understood, therefore, that this technology is not limited to the particular embodiments disclosed, but it is intended to cover modifications within the spirit and scope of the present technology. Numerous characteristics and advantages have been set forth in the foregoing description, together with details of structure and function. The novel features are pointed out in the appended claims. The disclosure, however, is illustrative only and changes may be made in detail, especially in matters of shape, size, and arrangement of parts, within the principle of invention, to the full extent indicated by the broad general meaning of the terms in which the appended claims are expressed.

What is claimed is:

1. A drainage system comprising:
a portable ergonomic carrier and
a disposable collector, said collector further comprising at least one suction mechanism within said disposable collector, and further comprising at least one valve mechanism within said disposable collector;
wherein said disposable collector is engagingly mated and coupled demountably into said portable ergonomic carrier.

2. The system of claim 1, further comprising, at least one electric motor mounted on said portable ergonomic carrier.

3. The system of claim 1, further comprising at least one electronic driver mounted on said portable ergonomic carrier.

4. The system of claim 1, further comprising at least one battery mounted on said portable ergonomic carrier.

5. The system of claim 1, further comprising at least one filter mechanism within said disposable collector.

6. The system of claim 1, further comprising at least one collecting chamber within said disposable collector.

7. The system of claim 1, where said inlet member is demountably joined to a thorax tube.

8. The system of claim 1, where said system used with a thoracotomy to extract fluids and gases from a human body.

9. The system of claim 1, where said carrier contains an at least one attachment mechanism to the human body.

10. A disposable drainage apparatus comprising:
at least one collector chamber, said collector chamber further comprising at least one inlet member and at least one outlet member;
at least one suction mechanism, said suction mechanism adapted to said inlet and outlet member, said suction mechanism further adapted to be engagingly mated and coupled demountably to at least one external moving shaft;
at least one valve mechanism contained within said suction mechanism and at least one filter mechanism;
wherein said suction mechanism, valve mechanism and filter mechanism are contained within said collector chamber.

11. The disposable apparatus of claim 10, where said collection chamber is further divided into smaller collection chambers.

12. The disposable apparatus of claim 10, where said filter mechanism used liquids is selected from the group consisting of water, water with additives, alcohols, gels and oils.

13. The disposable apparatus of claim 10, is made from transparent plastic selected from the group consisting of polycarbonate, polymethylmethacrylate, polystyrene, styrene-acrylonitrile copolymer, and poly vinyl chloride.

14. The disposable apparatus of claim 10, where said inlet member is demountably joined to a thorax tube.

15. The disposable apparatus of claim 10, where said apparatus is used with a thoracotomy to extract fluids and gases from a human body.

16. A portable drainage carrier apparatus comprising:
at least one wall determined to configure at least one cavity, said cavity to be engagingly mated and coupled demountably to at least one disposable apparatus;
at least a motor, said motor further comprising at least a moving member responsive to said motor;
at least one electronic driver, and
at least one battery, said battery for energy supply to said motor and to said electronic driver;
wherein said motor and battery are disposed and mounted integrally to said wall determined to configure a cavity.

17. The portable drainage carrier apparatus of claim 16, where said carrier contains an at least one LED energized by said battery.

18. The portable drainage carrier apparatus of claim 16, where said carrier contains an at least one attachment mechanism to the human body.
